SEQUENCE LISTING (5 sequences)

SEQ ID NO: 1 - protein, 21 aa, Human
MEVGWYRSPFSRVVHLYRNGK

SEQ ID NO: 2 - protein, 13 aa, Artificial Sequence
  Synthetic
VHFFGNIVTPRTP

SEQ ID NO: 3 - protein, 13 aa, Artificial Sequence
  Synthetic
VHFFANIVTPRTP

SEQ ID NO: 4 - protein, 13 aa, Artificial Sequence
  Synthetic
VHFFKNIVTARTP

SEQ ID NO: 5 - protein, 21 aa, Artificial Sequence
  Synthetic
MEVGWYRSPFDRVVHLYRNGK

(19) United States
(12) Patent Application Publication
(10) Pub. No.: US 2011/0033488 A1
(43) Pub. Date: Feb. 10, 2011

(54) AGENTS AND METHODS FOR TREATMENT OF ANXIETY DISORDERS

(75) Inventors: Michal Eisenbach-Schwartz, Rehovot (IL); Gil M. Lewitus, Rehovot (IL)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(73) Assignee: Yeda Research and Development Co. Ltd., Rehovot (IL)

(21) Appl. No.: 12/988,053

(22) PCT Filed: Apr. 16, 2009
(86) PCT No.: PCT/IL2009/000409

§ 371 Date: Nov. 1, 2010

Related U.S. Application Data
(60) Provisional application No. 61/045,004, filed on Apr. 15, 2008.

(51) Int. Cl. A61K 39/00; A61K 38/16; A61K 35/14; A61P 25/22; A61P 25/00

(52) U.S. Cl. 424/185.1; 514/17.7; 424/93.71

(57) ABSTRACT

Peptides derived from CNS-specific antigens, altered peptide ligands (APL) analogues of said peptides, T cells activated by such peptides, poly-YE, and any combination of said agents are useful for prevention, treatment and/or alleviation of anxiety disorders, particularly post-traumatic stress disorder, and for restoring BDNF levels in the brain of an individual after reduction of BDNF expression induced by stress.

### FIELD OF THE INVENTION

The present invention relates to agents and methods for prevention, treatment and/or alleviation of anxiety disorders, particularly post-traumatic stress disorder (PTSD).

### BACKGROUND OF THE INVENTION

The response to stress is characterized by both emotional and physical manifestations, often leading to activation of various physiological systems. This evolutionary adaptive response endows the organism with the ability to deal with the stressor by temporarily adapting the body's homeostasis to the novel situation. These stress response mechanisms are well regulated and, in the absence of pathology, enable the return to normal homeostasis when the source of stress is removed. However, when homeostasis is not restored and maintained, long lasting changes can arise; in humans, these changes may lead to post-traumatic stress disorder (PTSD).

One of the physiological systems affected by stress is the immune system. The effects of stress on the immune system are highly variable. While in some cases, stress can suppress immunity, thereby increasing susceptibility to infection and cancer, in other situations, stress is thought to aggravate inflammatory diseases such as autoimmune disease. The main reason for this apparent dichotomy may be the body's different reactions to different types of stressor and different durations of stress (acute versus chronic). Less studied, however, is how the immune system helps to restore homeostasis.

Recent studies have shown that acute stress mobilizes lymphocytes from the blood to target organs such as the skin and lung (Dhabhar and McEwen, 1996). This stress-induced mobilization may represent an adaptive response that could increase immune surveillance and immune responses in organs to which lymphocytes traffic during stress.

C57BL/6J and BALB/c mice differ in their behavioral, endocrine and immune response following psychological stress. Thus, these two strains exhibit different changes in their immune response following stress (Shanks and Kusnecov, 1998). Exposure of BALB/c mice to stress in close proximity to vaccination with a specific antigen enhances the level of primary antigen-specific IgM and IgG antibodies; however, stress has no effect in C57BL/6J mice (Shanks and Kusnecov, 1998). Moreover, acute stress enhances delayed-type hypersensitivity in BALB/c but not in C57BL/6J mice (Flint and Tinkle, 2001). These strain differences have been mainly attributed to the response of the hypothalamic-pituitary-adrenocortical (HPA) axis to stress. C57BL/6J mice were reported to be relatively stress resistant, and although they have basal corticosterone levels similar to BALB/c mice, they produce lower concentrations of adrenocorticotropic hormone (ACTH) in response to acute stressors (Anisman et al., 1998).

Activation of the HPA axis can modulate immune activation (Pawlikowski et al., 1994; Kruger, 1996). Conversely, cytokines can influence HPA activity (Turnbull and Rivier, 1999). As acute stress can occur as part of daily activities, all the above adaptations may comprise a mechanism to maintain physiological homeostasis.

Recently, data from our laboratory have suggested that not only does stress affect the immune system, but no less importantly, the immune system affects the brain's stress response (Cohen et al., 2006). Using an animal model of a short-term exposure of mice to predator odor, we showed that T cell-deficient mice have a poor ability to adapt to psychological stress. Such exposure results in long-term effects on both behavioral and physiological functioning, reminiscent of PTSD in humans (Adamec et al., 1999; Cohen et al., 2003). Transgenic mice over expressing autoreactive T cells specific to the central nervous system (CNS)-related self-antigen, myelin basic protein (MBP), have a reduced incidence of PTSD-like symptoms relative to their matched controls. In contrast, transgenic mice over expressing T cells specific to a foreign antigen (ovalbumin) exhibit a higher incidence of PTSD-like symptoms (Cohen et al., 2006).

T cells are not only of importance in adapting to acute stress, but are also contributing to the maintenance of some cognitive function as well as to protection from mental dysfunction resulting from exposure to unbalanced levels of neurotransmitters (Kipnis et al., 2004). T cell-deficient mice have impaired cognition as assayed with the Morris Water Maze Learning and Memory Test (a hippocampal-dependent visuo-spatial learning and memory task). The cognitive impairment can be corrected by passive transfer of T cells from wild type mice. However, the exact mechanisms underlying the ability of the peripheral immune system to support and maintain mental and cognitive ability are not fully understood.

Specific nervous system (NS)-antigens, peptides derived therefrom, such modified peptides and T cells activated by the antigens or the peptides have been described in several patents and patent applications of the main inventor and her group as useful for preventing secondary neurodegeneration in injuries, diseases, disorders or conditions of the central nervous system (CNS) or peripheral nervous system (PNS). Reference is made in this respect to the publications WO 99/60021, WO 2002/055010, WO 2003/002602, WO 2005/046719, and WO 2005/055920 and their US counterpart applications, each and all of these applications being herein incorporated herewith in their entirety as if fully disclosed herein. However, none of these references demonstrates the activity in vivo of peptides derived from a CNS-antigen or a peptide obtained from modification of said peptide for treatment of psychological stress.

### SUMMARY OF THE INVENTION

In one aspect, the present invention relates to an agent selected from: (a) a peptide derived from a CNS-specific antigen; (b) an altered peptide ligand (APL) analogue of a peptide of (a); (c) T cells activated by a CNS peptide of (a) or an APL of (c); (d) poly-YE; and any combination of two or more agents of any of (a) to (d), for prevention, treatment and/or alleviation of an anxiety disorder.

In another aspect, the present invention relates to said agents (a) to (e) for restoring BDNF levels in the brain of an individual after reduction of BDNF expression induced by stress.

The present invention further provides methods for prevention, treatment and/or alleviation of an anxiety disorder and/or for restoring BDNF levels in the brain of an individual after reduction of BDNF expression induced by stress, which comprise administering to an individual in need thereof an effective amount of any of the agents (a) to (d) above or a combination of two or more agents of any of (a) to (d).

### BRIEF DESCRIPTION OF THE FIGURES

FIGS. 1A-E show that stress enhances immune trafficking in BALB/c mice. (1 Å) BALB/c mice were exposed to predator odor and were killed at 3, 24, 48 hours and 7 days thereafter; sections from their brains were analyzed by immunohistochemistry for the presence of CD3+ cells (T cells). Most of the CD3+ cells were found in the choroid plexus (Cpx). Bar graph indicates the average numbers of CD3+ cells per slice. Values represent means±S.E.M. (A one-way ANOVA indicated a significant difference between the different time points (F_{5,24}=14.55, P=0.0001); ***P<0.001 (Tukey-Kramer post hoc analysis); n=6 slices from 5 animals). (1B-i), a representative image of the Cpx of stressed mice 48 h after stress exposure. CD3+ cells are stained in red and marked by arrows. (1B-ii), representative image of the hypothalamus of a stressed mouse 48 hrs after exposure. CD3+ cells are indicated by arrows. (1C) Representative images of the Cpx and hypothalamus stained with anti-ICAM-1: from control mice (1C-i,iii) and mice 48 hrs after exposure to stress (1C-ii,iv), respectively. (1D) Analysis of ICAM-1 expression in the Cpx (left) and hypothalamus (right) of stressed BALB/c mice. Graph indicates the density of ICAM-1 in arbitrary units. Values represent means±S.E.M. (A one-way ANOVA indicated a significant difference between the different time points (F_{5.51}=7.96, P=0.0001); **<0.01, ***P<0.001 (Tukey-Kramer post hoc analysis); n=5). (1E) ICAM-1 expression in the Cpx (upper graph) and hypothalamus (lower graph) after s.c. injection of different concentrations of corticosterone (0.6, 6 and 60 mg/kg). Graph indicates density of ICAM-1 expression relative to ICAM-1 in vehicle-treated control mice. Values are means±S.E.M. (A one-way ANOVA indicated a significant difference between the different time points for each treatment. In the Cpx: 0.6 mg/kg (F_{3,34}=3.76, P=0.0194); 6 mg/kg (F_{3,31}=4.01, P=0.0159)*P<0.05; 60 mg/kg (F_{3,26}=3.64, P=0.0256)*P<0.05 (Tukey-Kramer post hoc analysis); n=5).

FIGS. 2A-C show in vitro expression of ICAM-1 on a choroid plexus cell line. The choroid plexus cells (ECACC # 00031626) were grown on cover slides and incubated with different concentrations of corticosterone (2A-2B) or TNF-α(2C) for 24 hrs, and then stained for ICAM-1. Graph indicates ICAM-1 expression relative to vehicle-treated control. (2A) Representative images of Cpx cells incubated with various concentrations of corticosterone: (i) control, (ii) 1 ng/ml and (iii) 20 ng/ml. Values are means±S.E.M. (A one-way ANOVA indicated significant difference between treatment groups. For coticosterone (F_{5,460}=46.60, P<0.0001), ***P<0.001; for TNF-α(F_{5,567}=71.72, P<0.0001) ***P<0.001 (Tukey-Kramer post hoc analysis) n=100 cells (average).

FIGS. 3A-C show that stress does not enhance immune surveillance in C57BL/6J mice. (1A) C57BL/6J mice, which have a reduced hypothalamic-pituitary-adrenal (HPA) response to stress, showed a transient infiltration of CD3+ cells to the Cpx after exposure to predator odor. Graph indicates the average numbers of CD3+ cells per slice. Values are means±S.E.M. (A one-way ANOVA indicated a significant difference between the different time points (F_{5,24}=7.06, P=0.0003); **P<0.01 (Tukey-Kramer post hoc analysis); n=6 slices from 5 animals). ICAM-1 expression in the Cpx (3B) and the hypothalamus (3C) of C57BL/6J mice at different time points after stress. Graph indicates ICAM-1 density in arbitrary units. Values are means±S.E.M. (A one-way ANOVA indicated a significant difference between the different time points. For Cpx: (F_{5,51}=4.88, P=0.001); for hypothalamus: (F_{5,49}=11.39, P=0.001); ***P<0.001, (Tukey-Kramer post hoc analysis); n=5).

FIGS. 4A-C show that expression of brain-derived neurotrophic factor (BDNF) in the hippocampus associates with adaptation to stress. (4A) A single 15 min exposure to predator odor resulted in a greater behavioral change in the C57BL/6J mice than in the BALB/c mice. C57BL/6J mice spent significantly less time than BALB/c mice exploring the open arms of the elevated plus-maze (left), and showed a stronger acoustic startle response than BALB/c mice (right). Values are means±S.E.M. (Student's t-test, *P<0.05. ***P<0.001) n=10). (4B) After exposure to the predator odor, sections of the hippocampus of BALB/c mice and C57BL/6.1 mice were stained for BDNF. A representative image of BDNF staining in the dentate gyrus (DG) of naïve mice (upper panels), and BALB/c and C57BL/6J mice 3 hrs (middle panels) and 7 days (lower panels) after exposure to the predator odor. (4C) Analysis of BDNF immunoreactivity in the DG of BALB/c (left) and C57BL/6J (right) mice at different time points after stress. Graph indicates BDNF density in arbitrary units. Values are means±S.E.M. (A one-way ANOVA indicated a significant difference between the different time points. For Balb/c: (F_{5,53}=9.93, P=0.0001); **P<0.01, ***P<0.001; for C57BL/6J: (F_{5,48}=4.06, P=0.0037); *P<0.05, **P<0.01, (Tukey-Kramer post hoc analysis); n=5).

FIGS. 5A-C show that vaccination with pMOG₃₅₋₅₅ reduces behavioral manifestations induced by acute stress in C57BL/6J mice. (5A) C57BL/6J mice were immunized with pMOG₃₅₋₅₅ or PBS emulsified with CFA 1 week before a 15 min exposure to predator odor. pMOG₃₅₋₅₅ immunized mice spent significantly more time exploring the open arms of the elevated plus-maze (left graph) and showed a reduced acoustic startle response versus PBS-treated mice (right graph). Values are means±S.E.M. (Student's t-test, *P<0.05) n=10. (5B) Representative image of BDNF staining in the DG of C57BL/6J mice immunized with pMOG₃₅₋₅₅ (right panels) or PBS emulsified in CFA (left panels) in naive mice (upper panels) or in mice 24 hrs (middle panels) and 7 days (lower panels) after exposure to predator odor. (5C) Quantification of BDNF immunoreactivity in the DG of pMOG₃₅₋₅₅ or PBS immunized C57BL/6J mice at 24 hrs and 7 days after exposure to predator odor. Note that the treated mice exhibited a reduction in the BDNF levels 24 hrs after the stress. However, 7 days after the stress exposure. the levels of BDNF in the mice treated with CFA alone were still low compared to the pMOG₃₅₋₅₅ immunized mice. Values represent means±S.E.M. (One-way ANOVA analysis indicated a significant difference between the different time points. (F_{5,53}=20.299, P=0.0001); **p<0.01, ***P<0.001, n=5).

FIGS. 6A-C show that vaccination with poly-YE reduces behavioral manifestations induced by acute stress in C57BL/6J mice. C57BL/6J mice were immunized with poly-YE (20 μg or 70μ) or PBS immediately after exposure to predator odor. Poly-YE (20 μg) immunized mice spent significantly more time exploring the open arms of the elevated plus-maze (FIG. 6A, upper graph) and showed a reduced acoustic startle response (FIG. 6A, lower graph) versus poly-YE (70 μg) or PBS-treated mice. (FIG. 6B) C57BL/6j mice were immunized with poly-YE (20μ) a week after exposure to predator odor. There was no significant difference between the poly-YE (20 μg) immunized mice exploration of the open arms of the elevated plus-maze (FIG. 6B, left graph) and reduction of acoustic startle response (FIG. 6B, right graph) versus PBS-treated mice. (FIG. 6C) Quantification of BDNF immunoreactivity in the DG of mice that were treated with poly-YE (20 μg) or PBS immediately after exposure to predator, at 7 days after exposure to predator odor.

### DETAILED DESCRIPTION OF THE INVENTION

The present invention relates to agents useful for prevention, treatment and/or alleviation of an anxiety disorder selected from: (a) a peptide derived from a CNS-specific antigen; (b) an altered peptide ligand (APL) analogue of a peptide of (a); (c) T cells activated by a CNS peptide of (a) or an APL of (c); (d) poly-YE; and any combination of two or more agents of any of (a) to (d).

In one preferred embodiment, the agent used in the invention is a peptide derived from a CNS-specific antigen. As used herein, the term “CNS-specific antigen” refers to an antigen specific to the CNS of an individual and the term “peptide derived from a CNS specific-antigen” relates to a peptide which sequence is comprised within the sequence of such a CNS-specific antigen For the purpose of this application, the term “peptide” includes also salts and chemical derivatives of said peptides such as esters, amides, etc.

The CNS specific-antigen may be selected from myelin oligodendrocyte glycoprotein (MOG), myelin basic protein (MBP), proteolipid protein (PLP), myelin-associated glycoprotein (MAG), oligodendrocyte-specific protein (OSP), myelin-oligodendrocytic basic protein (MOBP), S-100, β-amyloid, Thy-1, a peripheral myelin protein including P0, P2 and PMP22, neurotransmitter receptors, the protein Nogo including Nogo-A, Nogo-B and Nogo-C and the Nogo receptor.

In one preferred embodiment, the CNS-specific antigen is MOG and the peptide derived from MOG is preferably MOG₃₅₋₅₅ (SEQ ID NO: 1).

In another embodiment, the agent is an altered peptide ligand analogue of a peptide derived from a CNS specific-antigen, hereinafter “altered peptide” or “APL”, which is obtained by modification of a self-peptide derived from a CNS-specific antigen, which modification consists in the replacement of one or more amino acid residues of the self-peptide by different amino acid residues, said modified CNS peptide still being capable of recognizing the T-cell receptor recognized by the self-peptide but with less affinity. Thus, the altered peptides are derived from or cross-react with self-proteins that cause autoimmune diseases, but they are “safe”, i.e., do not induce autoimmune disease.

Generally, an altered synthetic peptide is a peptide comprising at least one nonameric core sequence which fits into the MS-relevant HLA-DR/DQ molecule and is flanked by 2-5 amino acids at its N- and C-termini, in which sequence one to three T-cell receptor (TCR) contact amino acid residues is/are substituted by different suitable amino acid residue(s), the resulting immunogenic epitope cluster altered in the TCR residue being capable of immunomodulating the potentially pathogenic T-cell response against the epitope without risk of exacerbation.

Examples of altered peptides that can be used in accordance with the invention include, without being limited to, altered peptides derived from MBP, MOG, OSP, MOBP, PLP, and MAG, as disclosed in US 2005/0037422, herewith incorporated by reference in its entirety as if fully disclosed herein. In a preferred embodiment, the altered peptides are derived from the residues 87-99 of human MBP, in which the lysine residue 91 is replaced by glycine (G91) (SEQ ID NO:2) or by alanine (A91) (SEQ ID NO:3) or the proline residue 96 is replaced by alanine (A96) (SEQ ID NO: 4). In another preferred embodiment, the altered peptide is MOG₃₅₋₅₅(D45) derived from the residues 35-55 of human MOG, in which the serine residue 45 is replaced by aspartate (MEVGWYRDPFSRVVHLYRNGK; SEQ ID NO: 5). Other altered peptides are envisaged by the invention such as the peptide analogues derived from the residues 86 to 99 of human MBP by alteration of positions 91, 95 or 97 as disclosed in U.S. Pat. No. 5,948,764, and the altered peptides analogues to Nogo and Nogo receptor-derived peptides as described in WO 03/002602, both publications herewith incorporated by reference in their entirety as if fully disclosed herein.

In another embodiment, the agent for use in the invention is T cells activated by the CNS-specific peptide or by the altered peptide used in the present invention. “Activated T cell” as used herein includes (i) T cells that have been activated by exposure to said peptide or altered peptide and (ii) progeny of such activated T cells. Alternatively, the T cell which has been previously exposed to the peptide may be activated by a mitogen, such as phytohemagglutinin (PHA) or concanavalin A. The T cells according to the invention are preferably autologous T cells, namely, obtained from the same individual to whom they are going to be administered after activation, but also envisaged is the use of allogeneic T cells from related donors, or HLA-matched or partially matched, semi-allogeneic or fully allogeneic donors.

In another embodiment, the active ingredient is poly-YE. Poly-YE or poly-Glu,Tyr is a non-pathogenic synthetic random copolymer composed of the two amino acids L-glutamic acid (Glu, E) and L-tyrosine (Tyr, Y). In one preferred embodiment, it is the copolymer poly-Glu⁵⁰Tyr⁵⁰ with an average length of 100 amino acids and a capacity to elicit strong immune response in certain mouse strains. Poly-YE was described in WO 03/002140 of the present applicant for preventing or inhibiting neuronal degeneration or for promoting nerve regeneration in the CNS or PNS, or for protecting CNS or PNS cells from glutamate toxicity. It was also disclosed in WO 2005/055920 of the present applicant for treatment of psychiatric disorders, but it has not been specifically tested in a model of PTSD as in the present application.

The peptides and T cells of the invention are for use in prevention, treatment and/or alleviation of anxiety disorders including phobic disorders, obsessive-compulsive disorder, post-traumatic stress disorder (PTSD), acute stress disorder and generalized anxiety disorder.

In a most preferred embodiment the anxiety disorder is post-traumatic stress disorder (PTSD), an anxiety disorder that can develop after exposure to a terrifying event or ordeal in which grave physical harm occurred or was threatened. Traumatic events that can trigger PTSD include violent personal assaults such as rape or mugging, natural or human-caused disasters, accidents, or military combat. PTSD can be extremely disabling.

In one preferred embodiment, the agent (a) to (e) according to the invention is useful for prevention of PTSD symptoms. In another embodiment, the agent is useful for treatment and/or alleviation of PTSD symptoms. In a further embodiment, the agent is useful for treatment of PTSD and may cause prevention or alleviation of the PTSD symptoms. The agent is preferably a CNS-specific peptide or an altered peptide. In one embodiment, the agent is the MOG peptide of SEQ ID NO: 1. In another embodiment, the agent is the altered MOG peptide of SEQ ID NO:5. In a further embodiment, the agent is poly-YE.

The invention further provides the use of an agent (a) to (e) as defined herein for the preparation of a pharmaceutical composition for prevention, treatment or alleviation of the symptoms of PTSD.

Pharmaceutical compositions for use in accordance with the present invention may be formulated in a conventional manner using one or more physiologically acceptable carriers or excipients. The carrier(s) must be “acceptable” in the sense of being compatible with the other ingredients of the composition and not deleterious to the recipient thereof. The term “carrier” refers to a diluent, adjuvant, excipient, or vehicle with which the therapeutic is administered.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes.

As will be evident to those skilled in the art, the therapeutic effect depends at times on the condition or disease to be treated, on the individual's age and health condition, on other physical parameters (e.g., gender, weight, etc.) of the individual, as well as on various other factors, e.g., whether the individual is taking other drugs, etc., and thus suitable doses and protocols of administration will be decided by the physician taking all these factors into consideration.

The invention will now be illustrated by the following non-limiting examples.

### EXAMPLES

Immune surveillance of specific organs such as the skin and lung, following acute stress, is part of the body's mechanism of defense. Here we demonstrate that infiltration of T cells to the brain is similarly needed to alleviate the negative physiological effects of psychological stress such as anxiety and acoustic startle response. According to the present invention, we show that short exposure of mice to a stressor (predator odor) enhanced T-cell infiltration to the brain, especially to the choroid plexus, and that this migration was associated with increased ICAM-1 expression by choroid plexus cells. Systemic administration of corticosterone could mimic the effects of psychological stress on ICAM-1 expression. Furthermore, we found that the ability to cope with the stress of the predator odor correlated with enhancement of T-cell trafficking to the brain and with the reversal of stress-induced down regulation of hippocampal BDNF expression. Vaccination with a CNS-related peptide (MOG₃₅₋₅₅) reduced the levels of anxiety and the acoustic startle response induced by the acute stress, and induced recovery of BDNF levels. These results may lead to development of a therapeutic vaccine to alleviate chronic consequences of acute psychological trauma, such as post-traumatic stress disorder.

### Materials and Methods.

(i) Animals. Adult wild-type mice of the BALB/c/OLA and C57BL/6J strains, all aged 8 to 12 weeks, were supplied and maintained under germ-free conditions by the Animal Breeding Center of The Weizmann Institute of Science (Rehovot, Israel). The mice were housed in a light- and temperature-controlled room and were matched for age in each experiment. All animals were handled according to the regulations formulated by the Institutional Animal Care and Use Committee.

(ii) Experimental Stress Paradigm. The mice to be tested (experimental group) were placed for 15 min on thoroughly soaked cat litter (used by a cat for 2 days and sifted for feces).

(iii) Behavioral Testing.

Elevated Plus-Maze. The maze used is a black opaque Perspex platform with four arms in the shape of a plus, elevated 78 cm above the ground, as described by File (File, 1993; Griebel et al., 1995). Each arm was 24 cm long and 7.5 cm wide. One pair of opposite arms was “closed”, and thus the arms were enclosed by 20.5 cm high Perspex walls on both sides and on the outer edges of the platform, while the other pair of arms was “open”, surrounded only by a 3 mm high Perspex lip, which served as a tactile guide for animals in the open areas. The apparatus was illuminated by dim red lighting that provided 40-60 lux in both the open and the closed arms. Mice were placed one at a time in the central platform for 5 min, facing different arms on different days according to a randomized sequence. Between test sessions, the maze was cleaned with an aqueous solution of 5% ethanol and dried thoroughly.

Five behavioral parameters were assessed: (1) time spent in the open arms; (2) time spent in the closed arms; (3) number of entries into the open arms; (4) number of entries into closed arms; (5) total number of entries into all arms. Mice were recorded as having entered an open or closed arm only when all four paws crossed the dividing line between the arms and central platform. The number of entries into any arm of the maze (total arm entries) was defined as “exploratory activity”.

Acoustic Startle Response. Pairs of mice were tested in startle chambers. The acoustic startle responses were measured in two ventilated startle chambers (SRLAB System; San Diego Instruments, San Diego, Calif.). Each chamber consisted of a Plexiglas cylinder resting on a platform inside a ventilated, sound-attenuated chamber. Movement of the animal inside the tube was detected by a piezoelectric accelerometer located below the frame. The amplitude of the acoustic startle response of the whole body to an acoustic pulse was defined as the average of 100 accelerometer readings, 100 ms each, collected from pulse onset. The readings (signals) were digitized and stored in a computer. To ensure consistent presentation, sound levels within each test chamber were routinely measured using a sound-level meter (Radio Shack, San Diego Instruments). An SR-LAB calibration unit was routinely used to ensure consistency of the stabilimeter sensitivity between test chambers and over time (Swerdlow and Geyer, 1998). Each startle session started with a 5-min acclimatization period to a background of 68 dB white noise; following habituation, 30 acoustic startle trial stimuli were presented (110 dB white noise of 40 ms duration with 30 or 45 sec inter-trial interval).

Rota-road treadmills. Motor strength and coordination were evaluated on the accelerating Ugo Basile Model 7650 Rota-rod apparatus (Ugo Basile, Camerio, Italy). Each mouse was placed on the cylinder, which increased rotation speed from 5 to 40 rpm over a 300s period. Mice were first given three trails to become acquainted with the Rota-rod apparatus before the test. For detection, a group of 5 mice were placed on the rotating rod before starting the accelerated program. The time each mouse remained in the rod was registered automatically. If the mouse remained on the rod for 300 s (top speed of the rod) the test was completed and scored as 300 s.

(iv) Choroid Plexus endothelial cell culture. RCP cells (ECACC # 00031626, a kind gift from Dr. A. Chen, Weizmann Institute) (Battle et al., 2000) were cultured at 37° C. under 95% air/5% CO2 in DMEM medium containing 10% FCS, 100 U/ml penicillin, 100 mg/ml streptomycin, 1% insulin-transferrin-selenium mix (Sigma-Aldrich) and 0.1% Epidermal Growth Factor (10 mg/ml). The culture medium was changed every 2 days.

Choroid plexus (CPx) cultures were treated for 24 hours with different doses of corticosterone (1 ng/ml, 5 ng/ml, 10 ng/ml, 20 ng/ml and 50 ng/ml) or with the cytokines TNF-a, IFN-γ, IL-16 and IL-10 (each cytokine was administered at 1 ng/ml, 10 ng/ml, 20 ng/ml, 50 ng/ml and 100 ng/ml).

(v) Corticosterone administration. Corticosterone (Sigma-Aldrich) was dissolved in polyethylene glycol 400 (PEG) (Sigma-Aldrich), and each mouse received a single s.c. injection of corticosterone (0.6 mg/kg, 6 mg/kg or 60 mg/kg in 0.01 ml PEG) or PEG alone.

(vi) Immunohistochemistry and tissue preparation. The animals were deeply anesthetized and perfused transcardially, first with PBS and then with 2.5% paraformaldehyde. Their brains were removed, postfixed overnight, and then equilibrated in phosphate-buffered 30% sucrose. Free-floating 30-μm longitudinal sections were collected on a freezing microtome (SM2000R; Leica Microsystems) and stored at 4° C. prior to immunohistochemistry.

For immunohistochemistry, coronal sections of the brain (30 μm) were treated with a blocking solution containing 20% horse serum (HS), 0.1% Triton X-100 except for sections to be stained for BDNF, for which the blocking solution contained 0.05% saponin (Sigma-Aldrich). Primary antibodies were applied in a humidified chamber at room temperature. Tissue sections were then labeled overnight with the following primary antibodies (Abs): rabbit anti-CD3 (Dako Cytomation), hamster anti-mouse CD54 (ICAM-1) (Chemicon), goat anti-mouse CD106 (VCAM-1) (R&D Systems), and rabbit anti-BDNF (Alomone Labs).

Secondary antibodies used for immunohistochemistry were Cy-3-conjugated donkey anti-rabbit, and Cy-3-conjugated donkey anti-goat. For ICAM-1 staining, biotin conjugated goat anti-hamster was applied for 1 hr, followed by streptavidin-Cy3 for 15 min. All secondary antibodies were purchased from Jackson ImmunoResearch Laboratories Inc. Control sections (not treated with primary antibody) were treated with secondary antibodies to distinguish specific staining from nonspecific staining or autofluorescent components. Sections were then washed with PBS and cover slipped in polyvinyl alcohol with diazabicylo-octane as an anti-fading agent.

CPx cultures grown on cover slips were washed with PBS, and fixed as described above. The fixed cells were then treated with a blocking solution containing 10% FCS, 2% bovine serum albumin, 1% glycine, and 0.1% Triton X-100 (Sigma-Aldrich), and stained with hamster anti-mouse CD54 (ICAM-1) (Chemicon).

(vii) Immunization. Adult mice were immunized with 100 μg pMOG₃₅₋₅₅ (MEVGWYRSPFSRVVHLYRNGK; SEQ ID NO: 1) (Schori et al., 2001; Lewitus et al., 2006), emulsified in an equal volume of CFA (Difco, Franklin Lakes, N.J.) containing Mycobacterium tuberculosis (0.5 mg/ml, Difco). The emulsion was injected s.c. at a single site in the flank. Control mice were injected with PBS emulsified with CFA.

(viii) Quantitation. BDNF and ICAM-1 immunoreactivity was quantified blindly with image Pro Plus 4.5 software (Media Cybernetics) (Ziv et al., 2006b).

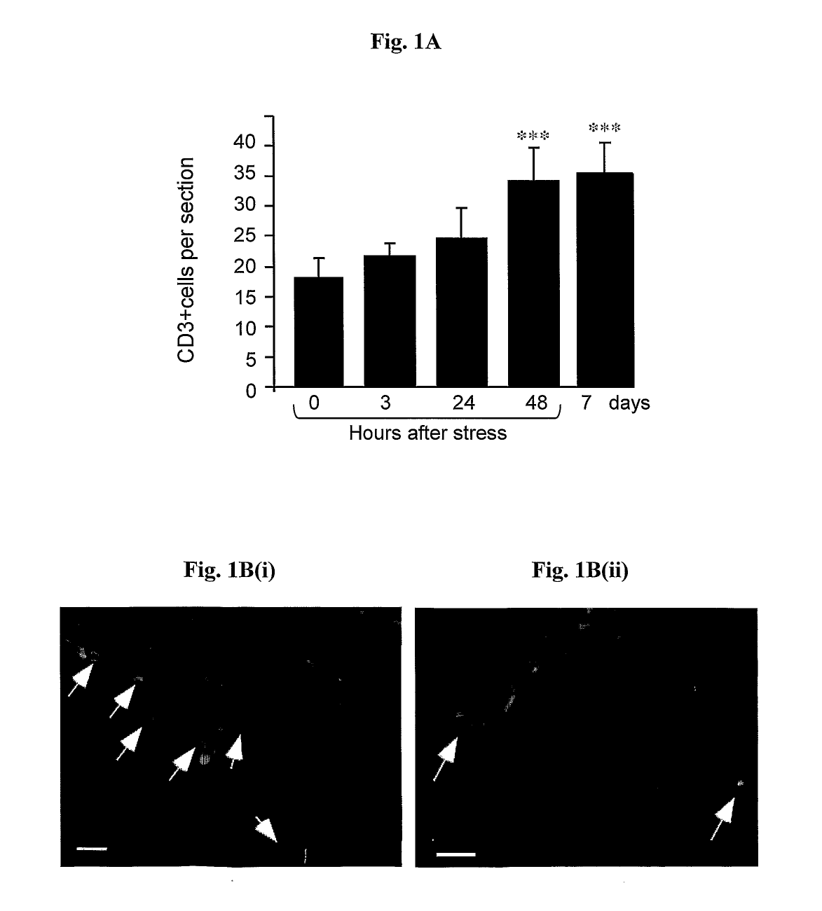

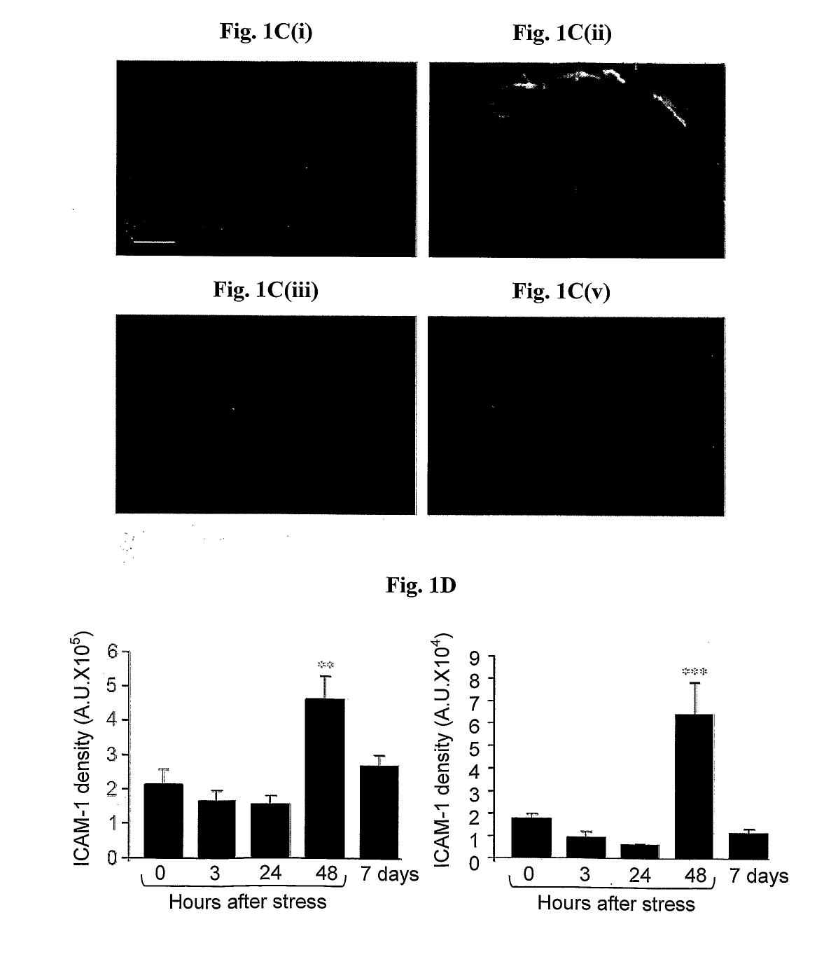

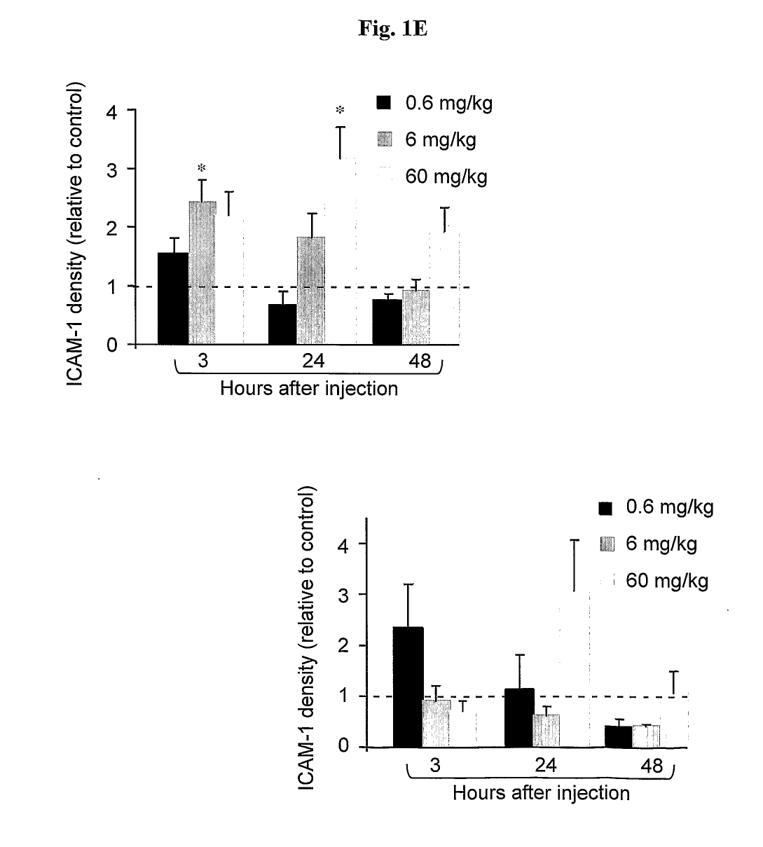

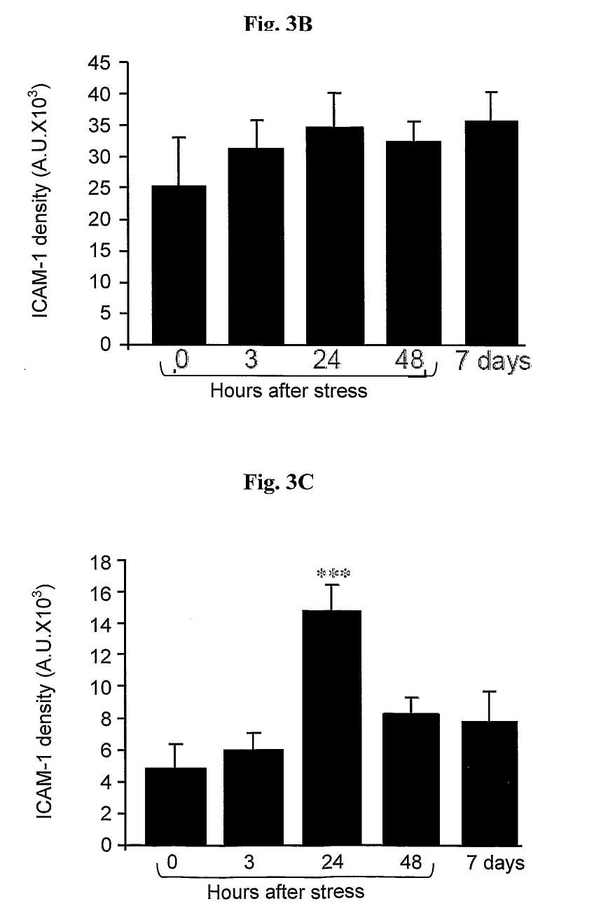

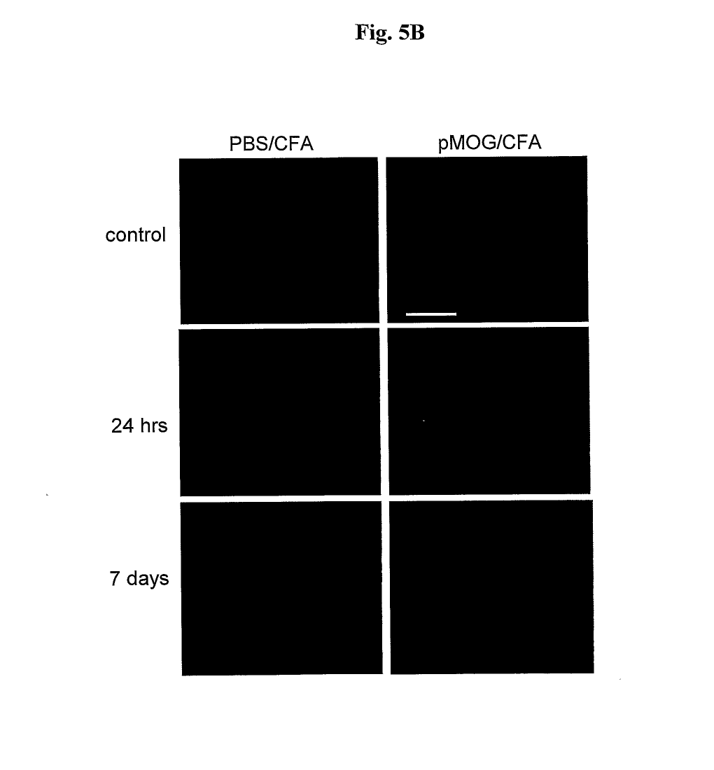

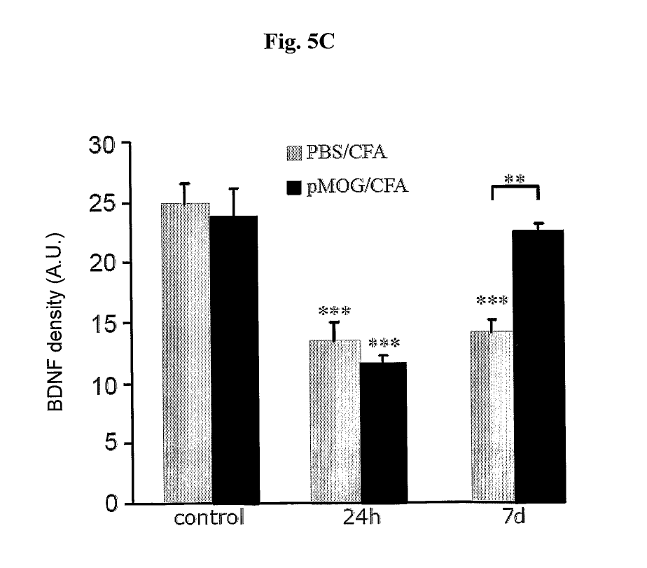

(ix) Statistical analysis. A two-tailed unpaired Student's t-test was used for analyses of the experiments presented in FIGS. 3C and 5A. The data from the experiments presented in FIGS. 1-5 were analyzed by ANOVA, and means were compared using the Tukey-Kramer post hoc analysis test for differences between individual means. Values that differed at p<0.05 were considered statistically significant. All data are represented as means±S.E.M.

### Example 1

### Recruitment of Lymphocytes to the CNS in Response to Predator Odor

Recently, we have shown that the ability to cope with psychological stress (predator odor) is dependent on the availability of CNS-specific T cells (Cohen et al., 2006). We have proposed that such autoreactive T cells are required at sites sensitive to stress and, accordingly, we suggested that stress may enhance T-cell recruitment to the brain in a manner similar to their recruitment to the other target organs (skin and lungs).

To investigate the recruitment of lymphocyte to the brain following exposure to predator odor we used BALB/c mice, previously shown to be less vulnerable to predator odor. The mice were exposed for 15 min to cat litter (predator odor) and their brains were excised at 3, 24, 48 hrs as well as 7 days after exposure, and analyzed by immunohistochemistry. We especially looked at lymphocyte accumulation in the choroid plexus (Cpx) surrounding the hippocampus as it forms the main T cells entry point to the CNS (FIG. 1B-i). Staining for CD3 (a marker for T cells) revealed that as early as 48 hrs after the exposure to stress there was a two-fold increase in the number of CD3+ cells in the Cpx of the stressed animals (34.2±2.47 average per slice in the stressed mice relative to 18.2±1.35 in unstressed controls) (FIG. 1A) and the numbers of these cells remained high 7 days after the stress, the latest time point that we tested. Furthermore, CD3+ cells were also seen in the hypothalamus, but in smaller numbers (FIG. 1B-ii). These results support our hypothesis that acute psychological stress induces an increase of brain surveillance by CD3+ cells.

To determine which adhesion molecules are involved in T-cell recruitment following stress, we stained brains from stressed mice for VCAM-1 and ICAM-1, the main adhesion molecules that are thought to be involved in CNS immune trafficking (Carrithers et al., 2000; Greenwood et al., 2002). Most of the ICAM-1 expression was observed in the Cpx and on the blood vessels (FIG. 1C). In BALB/c mice, there was slight but non-significant reduction of ICAM-1 expression in the Cpx after 3 hrs and 24 hrs, but by 48 hrs there was a transient significant up-regulation of ICAM-1 expression with a two-fold increase in expression. Similarly, in the hypothalamus, ICAM-1 expression peaked at 48 hrs (FIG. 1D). In contrast, VCAM-1 gave only weak staining, with no observed differences between the various groups; as a positive control for the staining high expression of VCAM-1 was observed in brains in the presence of an inflammatory response (data not shown). These results suggest that stress selectively upregulated ICAM-1 expression, and that ICAM-1 might be responsible for the enhanced accumulation of immune cells in the brain following an acute stress.

### Example 2

### Systemic Corticosterone Mimics the Effect of Stress on Lymphocyte Recruitment

It was previously shown that corticosterone elevation, in response to acute stress, is one of the hormonal mediators of stress-induced lymphocyte trafficking to peripheral tissues (Dhabhar and McEwen, 1999). Furthermore, cortisol was shown to upregulate LFA-1 (the ligand for ICAM-1) expression on lymphocytes following acute stress (Tarcic et al., 1995). Therefore, we wished to determine whether administration of exogenous corticosterone could mimic the effect of the stress on ICAM-1 expression in the brain.

To this end, we injected mice with corticosterone (0.6 mg/kg, 6 mg/kg and 60 mg/kg), or with the vehicle polyethylene glycol (PEG); brains were excised after 3, 24 or 48 hrs and stained for ICAM-1. The elevation and the timing of ICAM-1 expression in the Cpx were dependent on the corticosterone dosage. Three hours after administration of 0.6 mg/kg corticosterone, a slight but not significant elevation of ICAM-1 in the Cpx was seen. When an intermediate dosage of corticosterone (6 mg/kg) was administered, the elevation of ICAM-1 expression at 3 hrs was statistically significant, and when the highest dosage of corticosterone (60 mg/kg) was administered, the peak of ICAM-1 expression occurred 24 hrs after the injection (FIG. 1E). In contrast to the Cpx, no statistically significant effects were observed in the hypothalamus at any of the tested dosages (FIG. 1E).

These results suggest that the effect of stress on the expression of ICAM-1 is partly mediated by the elevation of corticosterone, and that different levels of coticosterone might differentially affect the expression of ICAM-1 in the Cpx and the hypothalamus.

### Example 3

### Corticosterone Induces ICAM-1 Expression by Choroid Plexus-Derived Cells

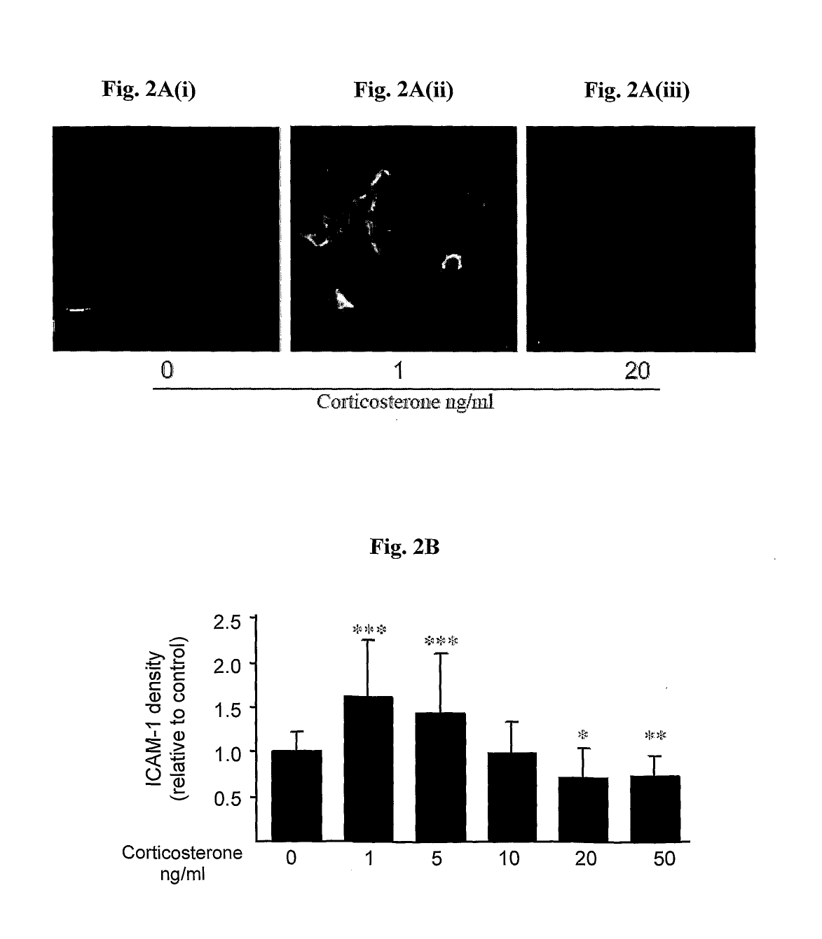

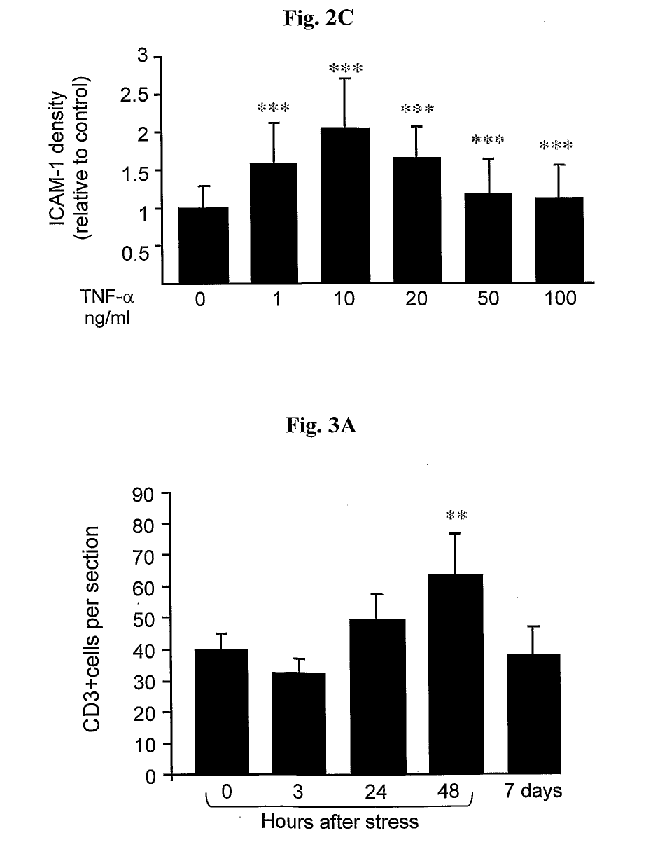

To further assess whether corticosterone can directly induce the elevation of ICAM-1, we incubated a choroid plexus cell line with different concentrations of corticosterone. At the lower doses of corticosterone (1 ng/ml and 5 ng/ml), ICAM-1 expression was significantly elevated, peaking at 1 ng/ml. At the higher doses (20 ng/ml and 50 ng/ml) ICAM-1 was significantly inhibited (FIGS. 2A-B). These results suggest that low levels of corticosterone induce, via ICAM-1, brain surveillance by immune cells, unlike high doses of corticosterone that are immune suppressive.

There is considerable evidence showing that acute psychological stress elevates plasma and brain levels of several cytokines, including TNF-α, IL-6, IL-10 and IFN-γ. Therefore, we also wished to determine whether these cytokines are involved in the regulation of ICAM-1 expression by cells in the Cpx.

We incubated choroid plexus cells with the different cytokines at several concentrations for 24 hrs, and subsequently stained for ICAM-1. TNF-α caused a significant elevation of ICAM-1 expression at all concentrations tested (FIG. 2C). IL-6 had no effect on ICAM-1 expression at any concentration tested (not shown). IFN-γ was inactive at the lower concentrations (1 ng and 10 ng); however, at the higher concentrations there was significant inhibition of ICAM-1 expression (not shown). IL-10 significantly reduced the levels of ICAM-1 at all concentrations tested (not shown). These results suggest that TNF-α might be an early stress signal in the brain.

### Example 4

### Ability to Cope with an Acute Stress Associates with Immune Surveillance

To further understand the functional association between the recruitment of the lymphocytes to the brain and the ability of the mice to adapt to the acute stress, we examined the C57BL/6J mice; this strain has a reduced HPA axis response to stress (Anisman et al., 1998) and reduced stress-induced delayed-type hypersensitivity. Before looking at the CD3+ cell recruitment and ICAM-1 expression, we verified that the C57BL/6J mice indeed have reduced ability to adapt to the predator odor compared to BALB/c mice (data not shown). In the C57BL/6J mice, we found a transient increase of CD3+ cells in the Cpx at 48 hrs after the exposure to the stressor (63.4±6.03 in the stressed mice relative to 39.73±2.41 in unstressed controls) (FIG. 3A). The expression of ICAM-1 in the Cpx was not significantly affected by the stress (FIG. 3B). It is important to note that the basal level of ICAM-1 was similar between the two strains (not shown) although the basal levels of CD3+ cells in the Cpx of C57BL/6J animals were higher than in BALB/c. The expression of ICAM-1 in the Cpx was slightly elevated by 24 hrs (although not to a statistically significant extent), but by 48 hrs returned to control levels. In the hypothalamus, the level of ICAM-1 was significantly higher at 24 hrs (FIG. 3B). These results further supported a strong association between ICAM-I expression in the Cpx and recruitment of CD3+ cells in response to acute stress.

### Example 5

### Immune Surveillance Controls Hippocampal BDNF

The above observation, together with a recent study showing the importance of the adaptive immune system in the ability to adapt to mental stress and in preventing a long lasting abnormal behavioral stress response (Cohen et al., 2006), lead us to propose that mouse strains (e.g., BALB/c) that can effectively recruit T cells in response to stress would be better able to cope with stress on a behavioral level.

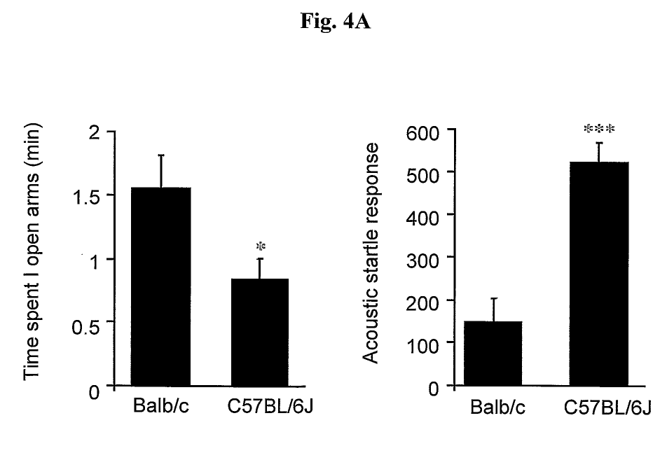

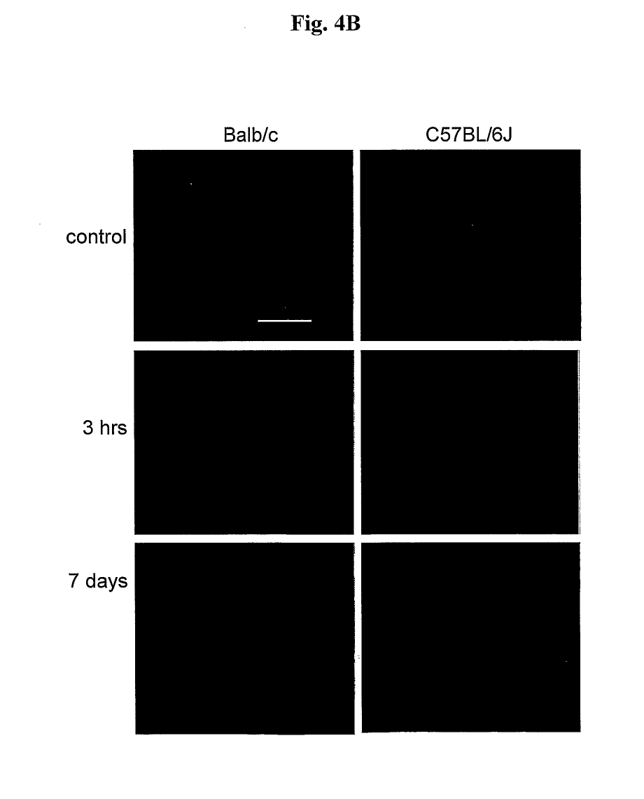

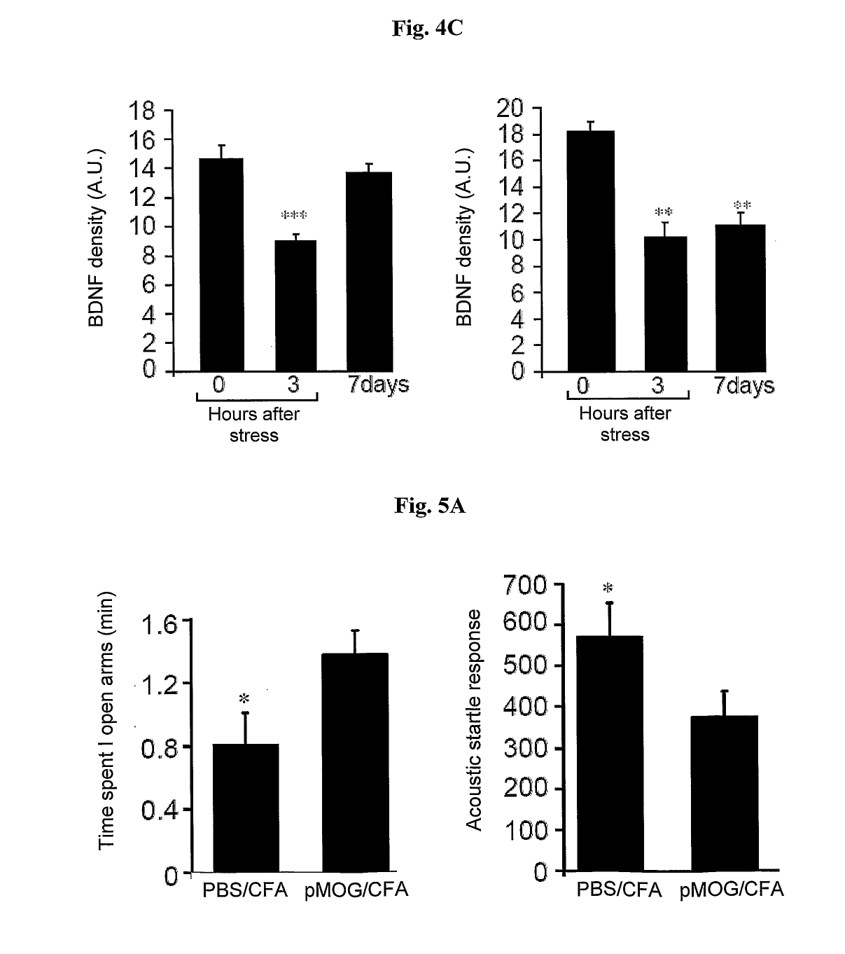

To test this hypothesis, we compared the long-term behavioral response to stress of BALB/c mice to that of C57BL/6J mice in the elevated plus maze and their acoustic startle response. A week after exposure to predator odor, C57BL/6J mice had higher anxiety levels than BALB/c mice as manifested in the time spent in the open arms of the elevated plus-maze as well as their greater acoustic startle response (FIG. 4A). Importantly, when mice from these two strains not exposed to stress were compared for their normal anxiety levels, the C57BL/6J mice did not show higher anxiety (Cohen et al., 2007). These results suggest that the BALB/c mice are better equipped to cope with stress than C57BL/6J mice, suggesting an association between enhanced immune surveillance and stress resilience.

Acute stress is known to reduce the expression of BDNF in the hippocampus (Smith et al., 1995). As T cells were shown to affect BDNF levels (Ziv et al., 2006b), we hypothesized that T-cell trafficking to the CNS would enable restoration of BDNF levels and increased ability to cope with stressful conditions. To correlate BDNF levels, we examined whether there are strain differences in the effect of stress on BDNF expression in the DG of the hippocampus (FIG. 4B). BALB/c and C57BL/6J mice were stained for BDNF at various time points following exposure to stress. In the BALB/c mice, there was a transient reduction of BDNF observed as early as 3 hrs after stress, but by day 7, BDNF levels returned to normal (FIG. 4C). In C57BL/6J mice, the reduction of BDNF was seen 3 hrs after the stress, yet in contrast to BALB/c, BDNF levels remained low even 7 days after stress application (FIG. 4C). These results suggest an association between stress-enhanced immune-surveillance, recovery of BDNF levels to normal, and adaptation to stress.

### Example 6

### Immunization with CNS-Related Peptide pMOG₃₅₋₅₅ Increases Ability to Cope with Stress

Immunization with a CNS-specific antigen was shown to rescue neurons from secondary damage by recruiting autoreactive T cells to the site of injury (Hauben et al., 2000). Therefore, we proposed that immunization with such an antigen might reduce the maladaptation to stress in C57BL/6J mice.

We immunized the mice with a MOG-derived peptide, pMOG₃₅₋₅₅, emulsified in CFA, 1 week before exposing the mice to predator odor. Mice were tested in the elevated plus maze and for the acoustic startle response 1 week after stress exposure. A significant difference was observed between the pMOG₃₅₋₅₅ immunized group and the control-injected mice. The immunized mice showed lower levels of anxiety, as measured by a weaker acoustic startle response as well as by the larger time spent in the open arms of the elevated plus-maze (FIG. 5A), and higher stimulatory activity (Table 1). To ensure that the reduced exploratory activity of the control mice was not due to reduced motor activity, 5 mice from each group underwent Rota-rod test. Both groups of mice spent equal time on the accelerating Rota-rod, further suggesting that the observed effect of the immunization was an outcome of reduced fearfulness (Table 1). These results suggest that manipulation leading to enhanced immune-surveillance of the brain can reduce maladaptation to stress.

TABLE 1
Immunization with pMOG₃₅₋₅₅ reduces behavioral manifestations
induced by acute stress in C57BL/6J mice

| Treatment | pMOG/CFA | PBS/CFA | Student's t-test |
|---|---|---|---|
| Parameters | | | |
| Time spent in the | 1.4 ± 0.1 | 0.8 ± 0.2 | t₁₇ = 2.23; |
| open arms (min) | | | P = 0.04 |
| Number of entries to | 3.3 ± 0.3 | 1.8 ± 1.5 | t₁₇ = 2.52; |
| the open arms | | | P = 0.02 |
| Exploratory activity | 17 ± 1.2 | 13.9 ± 0.7 | t₁₇ = 2.27; |
| | | | P = 0.03 |
| Acoustic startle | 344 ± 57.7 | 571.7 ± 82.1 | t₁₇ = 2.27; |
| amplitude | | | P = 0.03 |
| Rota rod (sec) | 234 ± 21.8 | 224 ± 4.5 | n.s. |

C57BL/6J mice were immunized with pMOG₃₅₋₅₅ or PBS emulsified with CFA one week before a 15 min exposure to predator odor. pMOG₃₅₋₅₅ immunized mice spent significantly more time exploring the open arms of the elevated plus-maze and showed a reduced acoustic startle response versus PBS-treated mice. Furthermore, there were no motor skil differences between the groups.
Values are means ± SEM.
n.s.—not significant.

The fact that we observed association between the BDNF expression and recruitment of immune cells to the brain, prompted us to examine whether the improved behavior induced by the immunization with pMOG₃₅₋₅₅ also resulted in restoration of BDNF levels. We therefore repeated the above experiment of immunizing C57BL/6J mice with pMOG₃₅₋₅₅ emulsified in CFA, or with CFA alone, and analyzed BDNF levels. The animals were exposed to predator odor 1 week after vaccination. The brains were tested for BDNF expression 24 hrs and 7 days after exposure to the stress (FIG. 5B). Unstressed animals, immunized with pMOG₃₅₋₅₅ or treated with CFA, were also analyzed on day 7 (14 days after immunization).

As expected, stress caused a reduction in BDNF levels, which was evident both at 24 hrs and 7 days. Yet, in the immunized animals, levels of BDNF were significantly restored 7 days following stress (FIG. 5C). No significant differences in BDNF levels were observed in the unstressed mice between the pMOG₃₅₋₅₅ immunized and the PBS-treated mice.

### Example 7

### Vaccination with Poly-YE Reduces Behavioral Manifestations Induced by Acute Stress in C57BL/6J Mice

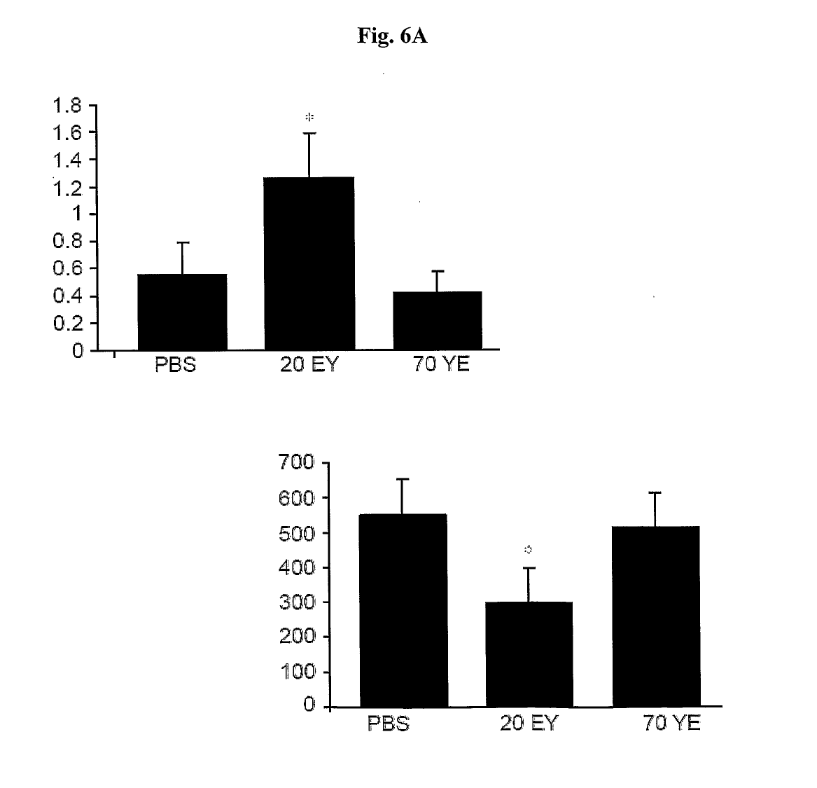

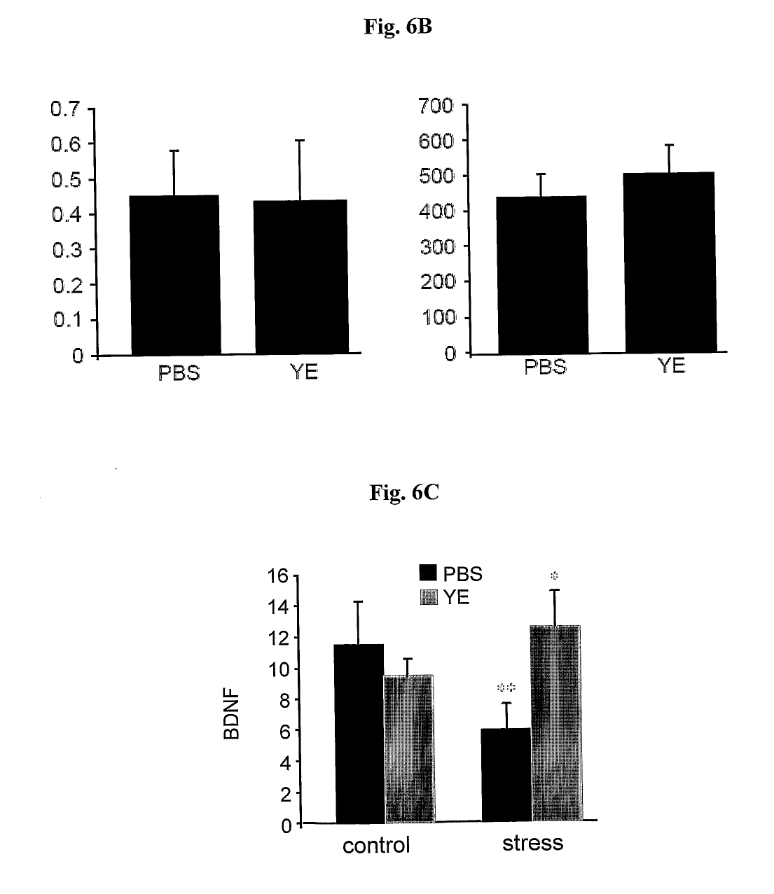

C57BL/6J mice were immunized with poly-YE/CFA (20 μg or 70 μg) or PBS/CFA immediately after exposure to predator odor. Poly-YE (20 μg) immunized mice spent significantly more time exploring the open arms of the elevated plus-maze (FIG. 6A, upper graph) and showed a reduced acoustic startle response (FIG. 6A, lower graph) versus poly-YE (70 μg) or PBS-treated mice. FIG. 6B depicts the results with C57BL/6j mice that were immunized with poly-YE (20μ) a week (7 days) after stress (exposure to predator odor). Note that no beneficial effect was observed. FIG. 6C shows quantification of BDNF immunoreactivity in the DG of mice that were treated with poly-YE (20 μg) or PBS immediately after exposure to predator, at 7 days after exposure to predator odor. Note, that 7 days after the stress exposure, there was elevation of BDNF in the mice treated with poly-YE

### Summary and Discussion

The results above show that trafficking of immune cells to the brain following acute stress is part of the defense mechanism against consequences of psychological stress. We also showed that immunization with CNS-derived peptide pMOG₃₅₋₅₅ reduced the delayed adverse behavioral effects of stress such as anxiety and the acoustic-startle response, by regulating levels of BDNF. The stress-induced brain surveillance by the immune system was associated with up regulation of ICAM-1 in the brain, especially in the choroids plexus and hypothalamus. A single injection of corticosterone could partially mimic the elevation of ICAM-1. Vaccination with the CNS-derived peptide MOG₃₅₋₅₅ reduced the long-lasting adverse behavioral effects, at least in part, by enhancing surveillance and restoring BDNF levels.

The enhanced trafficking of T cells to the brain was associated with enhanced ability to adapt to stress. Thus, for example, the BALB/c mice that demonstrated enhanced T cells recruitment to the brain, had lower levels of anxiety and reduced acoustic startle response relative to the C57BL/6J strain with reduced brain lymphocyte recruitment.

There are several lines of evidence suggesting a role for BDNF in the behavioral and cellular response to stress, and its pathophysiology (Duman et al., 2000). Acute stress such as immobilization (restraint stress) transiently down-regulates BDNF mRNA and protein expression in the hippocampus, especially in the DG (Smith et al., 1995). In our model of stress, we observed relationships between BDNF expression in the DG, the behavioral response to stress, and the degree of lymphocyte infiltration. In both strains, stress induced an immediate reduction of BDNF; however, by day 7, BDNF levels in the BALB/c mice were restored to the pre-stress levels, while in C57BL/6J mice, the levels of BDNF remained low.

Recently, it was shown (Kozlovsky et al., 2007) that rats that were exposed to predator odor exhibited a reduction in BDNF mRNA as well as protein levels in the hippocampus, similarly to our findings here. Additionally, a correlation was demonstrated between the behavioral response and BDNF levels. While rats with minimal behavioral response to stress, exhibited a transient reduction of BDNF levels in the hippocampus, in rats with extreme behavioral manifestations of stress, the reduction of BDNF in the DG was sustained for up to 7 days after stress exposure (Kozlovsky et al., 2007). These results further support the role of BDNF in the stress response. Moreover, as there is evidence for the involvement of the immune system in the maintenance of BDNF levels in the DG (Ziv et al., 2006), we suggest that one of the roles of the enhanced immune-surveillance induced by stress is to help in maintaining BDNF levels. To substantiate our hypothesis we immunized C57BL/6J (a strain with reduced ability to adapt to stress) mice with a CNS-derived peptide (MOG₃₅₋₅₅), a procedure which was shown to increase immune trafficking to the brain and was neuroprotective in several models of acute brain injury (Schori et al., 2001; Lewitus et al., 2006). The vaccination reduced the maladaptative behavior observed a week after stress exposure, and although the BDNF levels in the DG were reduced 24 hrs after the stress, by 7 days, the levels of BDNF were restored to the normal, pre-stress levels. These results further emphasize the importance of the immune system in brain homeostasis. It is important to note that the peptide used hereinabove, under certain protocols, can induce experimental autoimmune encephalomyelitis (EAE) in mice (Mendel et al., 1995). However, in the present invention, the protocol included only one immunization with the peptide (100 μg) without adding pertussis toxin used to induce EAE, and thus no EAE was induced. Importantly, we used this peptide in the present invention, as a proof of principle. For therapeutic purposes, weak agonist of self peptides such as altered peptide ligands, as well as additional peptides and carrier, are considered by the inventors (Hauben et al., 2001; Ziv et al., 2006a).

The results hereinabove illustrate a further manifestation of the complex interaction between the brain and the immune system; we showed that not only does stress influence immune trafficking to the brain but also that the immune system is highly active in maintaining brain homeostasis and in preventing the adverse effects of strong psychological stress. The present invention extends the role of ‘protective autoimmunity’ conceived some years ago by the main inventor, M. Schwartz, to include protection against mental stress, and further argues in favor of the importance of a distinction between a well-controlled immune response that takes place in the stressed brain, and a pathological immune response that occurs when immune response looses control such as in multiple sclerosis. According to this view, trafficking of T cells in response to stress is a desirable response and is amenable to boosting. Thus, PTSD in humans might be a reflection of insufficient or untimely recruitment of the immune system. Recognizing that the systemic immune system is a factor in containing mental stress offers new directions for the development of a therapy for stress-induced pathologies such as PTSD and depression, in the form of T cell-based vaccination, which increases the body's physiological ability to cope with stress.

### REFERENCES

Adamec, R. E., P. Burton, et al. (1999). “Unilateral block of NMDA receptors in the amygdala prevents predator stress-induced lasting increases in anxiety-like behavior and unconditioned startle—effective hemisphere depends on the behavior.” Physiol Behav 65(4-5): 739-51.
Anisman, H., S. Lacosta, et al. (1998). “Stressor-induced corticotropin-releasing hormone, bombesin, ACTH and corticosterone variations in strains of mice differentially responsive to stressors.” Stress 2(3): 209-20.
Battle, T., L. Preisser, et al. (2000). “Vasopressin V1a receptor signaling in a rat choroid plexus cell line.” Biochem Biophys Res Commun 275(2): 322-7.
Carrithers, M. D., I. Visintin, et al. (2000). “Differential adhesion molecule requirements for immune surveillance and inflammatory recruitment.” Brain 123 (Pt 6): 1092-101.
Cohen, H., A. B. Geva, et al. (2007). “Post-traumatic stress behavioural responses in inbred mouse strains: can genetic predisposition explain phenotypic vulnerability?” Int J Neuropsychopharmacol: 1-19.
Cohen, H., Y. Ziv, M. Cardon, Z. Kaplan, M. Mater, Y. Gidron, M. Schwartz, J. Kipnis. (2006). “Maladaptation to mental stress mitigated by the adaptive immune system via depletion of naturally occurring regulatory CD4+CD25+ cells.” J Neurobiol 66(6): 552-63.
Cohen, H., J. Zohar, et al. (2003). “The relevance of differential response to trauma in an animal model of posttraumatic stress disorder.” Biol Psychiatry 53(6): 463-73.
Dhabhar, F. S. and B. S. McEwen (1996). “Stress-induced enhancement of antigen-specific cell-mediated immunity.” J Immunol 156(7): 2608-15.
Dhabhar, F. S. and B. S. McEwen (1999). “Enhancing versus suppressive effects of stress hormones on skin immune function.” Proc Natl Acad Sci USA 96(3): 1059-64.
File, S. E. (1993). “The interplay of learning and anxiety in the elevated plus-maze.” Behav Brain Res 58(1-2): 199-202.
Flint, M. S. and S. S. Tinkle (2001). “C57BL/6 mice are resistant to acute restraint modulation of cutaneous hypersensitivity.” Toxicol Sci 62(2): 250-6.
Greenwood, J., S. Etienne-Manneville, et al. (2002). “Lymphocyte migration into the central nervous system: implication of ICAM-1 signalling at the blood-brain barrier.” Vascul Pharmacol 38(6): 315-22.
Griebel, G., D.C. Blanchard, et al. (1995). “A model of ‘antipredator’ defense in Swiss-Webster mice: effects of benzodiazepine receptor ligands with different intrinsic activities.” Behav Pharmacol 6(7): 732-745.
Hauben, E., E. Agranov, et al. (2001). “Posttraumatic therapeutic vaccination with modified myelin self-antigen prevents complete paralysis while avoiding autoimmune disease.” J Clin Invest 108(4): 591-9.
Hauben, E., O. Butovsky, et al. (2000). “Passive or active immunization with myelin basic protein promotes recovery from spinal cord contusion.” J Neurosci 20(17): 6421-30.
Kipnis, J., H. Cohen, et al. (2004). “T cell deficiency leads to cognitive dysfunction: implications for therapeutic vaccination for schizophrenia and other psychiatric conditions.” Proc Natl Acad Sci USA 101(21): 8180-5.
Kozlovsky, N., M. A. Matar, et al. (2007). “Long-term down-regulation of BDNF mRNA in rat hippocampal CA1 subregion correlates with PTSD-like behavioural stress response.” Int J Neuropsychopharmacol: 1-18.
Kruger, T. E. (1996). “Immunomodulation of peripheral lymphocytes by hormones of the hypothalamus-pituitary-thyroid axis.” Adv Neuroimmunol 6(4): 387-95.
Lewitus, G. M., J. Kipnis, et al. (2006). “Neuroprotection induced by mucosal tolerance is epitope-dependent: conflicting effects in different strains.” J Neuroimmunol 175(1-2): 31-8.
Mendel, I., N. Kerlero de Rosbo, et al. (1995). “A myelin oligodendrocyte glycoprotein peptide induces typical chronic experimental autoimmune encephalomyelitis in H-2b mice: fine specificity and T cell receptor V beta expression of encephalitogenic T cells.” Eur J Immunol 25(7): 1951-9.
Pawlikowski, M., H. Stepien, et al. (1994). “Hypothalamic-pituitary-thyroid axis and the immune system.” Neuroimmunomodulation 1(3): 149-52.
Schori, H., J. Kipnis, et al. (2001). “Vaccination for protection of retinal ganglion cells against death from glutamate cytotoxicity and ocular hypertension: implications for glaucoma.” Proc Natl Acad Sci USA 98(6): 3398-403.
Shanks, N. and A. W. Kusnecov (1998). “Differential immune reactivity to stress in BALB/cByJ and C57BL/6J mice: in vivo dependence on macrophages.” Physiol Behav 65(1): 95-103.
Swerdlow, N. R. and M. A. Geyer (1998). “Using an animal model of deficient sensorimotor gating to study the pathophysiology and new treatments of schizophrenia.” Schizophr Bull 24(2): 285-301.
Tarcic, N., G. Levitan, et al. (1995). “Restraint stress-induced changes in lymphocyte subsets and the expression of adhesion molecules.” Neuroimmunomodulation 2(5): 249-57.
Turnbull, A. V. and C. L. Rivier (1999). “Regulation of the hypothalamic-pituitary-adrenal axis by cytokines: actions and mechanisms of action.” Physiol Rev 79(1): 1-71.
Ziv, Y., H. Avidan, et al. (2006a). “Synergy between immune cells and adult neural stem/progenitor cells promotes functional recovery from spinal cord injury.” Proc Natl Acad Sci USA 103(35): 13174-9.
Ziv, Y., N. Ron, et al. (2006b). “Immune cells contribute to the maintenance of neurogenesis and spatial learning abilities in adulthood.” Nat Neurosci 9(2): 268-75.

What is claimed:

1. (canceled)

2. The method according to claim 8, wherein said agent is a peptide derived from a CNS-specific antigen selected from the group consisting of myelin oligodendrocyte glycoprotein (MOG), myelin basic protein (MBP), proteolipid protein (PLP), myelin-associated glycoprotein (MAG), S-100, β-amyloid, Thy-1, a peripheral myelin protein including P0, P2 and PMP22, neurotransmitter receptors, the protein Nogo including Nogo-A, Nogo-B and Nogo-C and the Nogo receptor, and an APL analogue of said peptide.

3. The method according to claim 2, wherein said peptide is derived from the CNS-specific antigen MOG.

4. The method according to claim 3, wherein said peptide is MOG₃₅₋₅₅ (SEQ ID NO: 1).

5. The method according to claim 2, wherein said peptide is an APL analogue of said peptide selected from the group consisting of the peptides MBP₈₇₋₉₉ (G91) (SEQ ID NO:2), MBP₈₇₋₉₉ (A91) (SEQ ID NO:3), MBP₈₇₋₉₉ (A96) (SEQ ID NO:4) and MOG₃₅₋₅₅ (D45) (SEQ ID NO:5).

6. The method according to claim 8, wherein said anxiety disorder is post-traumatic stress disorder.

7. (canceled)

8. A method for prevention, treatment and/or alleviation of an anxiety disorder, comprising administering to an individual in need thereof an effective amount of an agent selected from the group consisting of:
(a) peptide derived from a CNS-specific antigen;
(b) an altered peptide ligand (APL) analogue of a peptide of (a);
(c) T cells activated by a peptide of (a) or (b);
(d) poly-YE; and
(e) any combination of (a)-(d).

9. (canceled)

10. A method for restoring BDNF levels in the brain of an individual after reduction of BDNF expression induced by stress, which comprises administering to an individual in need thereof an effective amount of an agent selected from the group consisting of:
(a) a peptide derived from a CNS-specific antigen;
(b) an altered peptide ligand (APL) analogue of a peptide of (a);
(c) (d) T cells activated by a peptide of (a) or (b);
(d) poly-YE; and
(e) any combination of (a)-(d).

11. A method for treatment and/or alleviation of an anxiety disorder, comprising administering to an individual in need thereof an effective amount of an agent selected from the group consisting of:
(a) peptide derived from a CNS-specific antigen;
(b) an altered peptide ligand (APL) analogue of a peptide of (a);
(c) T cells activated by a peptide of (a) or (b);
(d) poly-YE; and
(e) any combination of (a)-(d).

12. The method according to claim 11, wherein said agent is a peptide derived from a CNS-specific antigen selected from the group consisting of myelin oligodendrocyte glycoprotein (MOG), myelin basic protein (MBP), proteolipid protein (PLP), myelin-associated glycoprotein (MAG), S-100, β-amyloid, Thy-1, a peripheral myelin protein including P0, P2 and PMP22, neurotransmitter receptors, the protein Nogo including Nogo-A, Nogo-B and Nogo-C and the Nogo receptor, or an APL analogue of said peptide.

13. The method according to claim 12, wherein said peptide is derived from the CNS-specific antigen MOG.

14. The method according to claim 13, wherein said peptide is MOG35-55 (SEQ ID NO: 1).

15. The method according to claim 12, wherein said peptide is an APL selected from the group consisting of the peptides MBP87-99 (G91) (SEQ ID NO:2), MBP87-99 (A91) (SEQ ID NO:3), MBP87-99 (A96) (SEQ ID NO:4) and MOG35-55 (D45) (SEQ ID NO:5).

16. The method according to claim 11, wherein said anxiety disorder is post-traumatic stress disorder.
